(19) United States
(12) Patent Application Publication
(10) Pub. No.: US 2026/0337538 A1
(43) Pub. Date: Oct. 29, 2026

(54) CELL CULTURE INSERT

(71) Applicant: Danmarks Tekniske Universitet, Kongens Lyngby (DK)

(72) Inventors: Alexander Jönsson, Kongens Lyngby (DK); Hakan Gürbüs, Kongens Lyngby (DK); Martin Dufva, Kongens Lyngby (DK)

(21) Appl. No.: 19/115,994

(22) PCT Filed: Sep. 29, 2023
(86) PCT No.: PCT/EP2023/077005

§ 371 Date: Mar. 27, 2025

(30) Foreign Application Priority Data
Sep. 29, 2022 (EP) 22198805.8

(51) Int. Cl. C12M 1/12

(52) U.S. Cl. CPC C12M 25/04

(57) ABSTRACT

The invention regards a cell culture insert comprising a chamber defined by at least one side wall and a first end wall, wherein the first end wall has a thickness equal to or below 500 μm and comprises at least one opening, wherein the at least one opening is defined by an opening perimeter, the perimeter having a surface configured for supporting a suspended membrane at the edges of the membrane by surface tension.

### TECHNICAL FIELD

The present invention relates to a cell culture insert, a kit of parts comprising the insert, a method of producing the insert, and a method of forming a suspended membrane for supporting biomaterials using the insert.

### BACKGROUND

Cell culture ware is a basic tool in biological laboratories and industries, and covers a range of standardised products such as flasks, dishes, and microtiter plates. The cell culture ware is typically made of plastics such a polystyrene and polycarbonate, and the culturing of the cells may be done directly on the plastic surface. To further improve the adhesion and support the viability of the biomaterial, the plastic surface may be coated with a biopolymer for e.g. improving the cell fate.

There is an increasing interest in cell culture ware specialized to support novel biomaterials, barrier cell models, and cell culture modalities including co-cultures. For these purposes, cell culture ware where the cell culture volume is divided into two or more compartments are advantageous.

Transwell® inserts in microtiter plate wells are known to provide a cell culture platform with multiple compartments. A Transwell® insert comprises a porous polymeric membrane bottom on which the cells may grow. The insert is placed in a microtiter plate well, such that the microtiter plate well is divided into a top and bottom part fluidly connected via the porous membrane. Accordingly, cells may be grown on the top and/or on the bottom of the membrane and form barriers or co-cultures. It also follows that multiple Transwell inserts may be stacked within a microtiter plate well to increase the co-culture capability.

However, the porous polymeric membrane of the Transwell® inserts has limited porosity, and is thus not suitable for modelling 3D tissue. Hence, to model 3D tissue the membrane and/or well may be coated with a 3D matrix, such as a hydrogel made of extracellular matrix protein similar to those found in tissue. For example, cells may be casted in a hydrogel layer casted on the membrane of the Transwell® insert. Since hydrogel layers are fragile and brittle, the hydrogel layer may be casted with a high thickness.

Despite the advances, there is a need for improved cell culture ware, especially platforms supporting novel biomaterials, barrier cell models, and other cell culture modalities.

### SUMMARY

The present disclosure relates to a cell culture insert, which is particularly suitable for supporting novel biomaterials, and which facilitates more reliable, flexible, and efficient testing of barrier cell models, and cell culture modalities. Specifically, the cell culture insert provides a reliable and flexible framework configured for supporting a suspended membrane with a higher similarity to real 3D tissue, and with improved possibility for cell movement and tissue remodelling.

The cell culture insert comprises a chamber with a first end wall comprising one or more openings, and where the thickness of the first end wall may be flexibly and reliably controlled. The precisely controlled thickness facilitates that the one or more openings are configured to comprise a suspended membrane layer, where the thickness may also be flexibly and reliably controlled, specifically with a controlled thickness below 500 μm, particularly below 300, 250, or 200 μm. Specifically, the one or more openings may be configured to comprise a suspended hydrogel film or membrane. Thus, only the opening perimeter is in contact with the suspended membrane, and the suspended membrane is supported at the lateral edges of the membrane by surface tension, optionally without any adhesives or glue, thereby facilitating that the thickness of the suspended membrane may be precisely controlled via the dimensions of the opening perimeter. The dimensions of the opening perimeter may be determined by the thickness of the first end wall, and thus the first end wall is advantageously essentially planar with a uniform thickness, particularly in the vicinity of the opening, such that the height of the perimeter may be similar to a predefined thickness of the first end wall. A thin suspended membrane has a more efficient mass transport for cell culturing and may have improved porosity and mechanical properties despite the low thickness. Accordingly, the membrane may have higher similarity to 3D tissue and be particularly efficient for testing of barrier cell models, and cell culture modalities.

A first aspect of the disclosure relates to a cell culture insert comprising a chamber defined by at least one side wall and a first end wall, wherein the first end wall has a thickness equal to or below 500 μm and comprises at least one opening.

In a preferred embodiment of the first aspect, the at least one opening is defined by an opening perimeter, the perimeter having a surface configured for supporting a suspended membrane at the edges of the membrane by surface tension, and wherein the first end wall is essentially planar with a uniform thickness. Accordingly, the perimeter surface may be configured for supporting both the formation of the suspended membrane, as well as the formed suspended membrane.

A second aspect of the disclosure relates to a method of producing the cell culture insert according to the first aspect by a fabrication technique selected from the group consisting of: injection molding, 3D printing, casting, and thermoforming, and a cutting process selected from the group consisting of: laser ablation, drilling, milling, punching, and optionally casting a suspended membrane within the at least one opening.

In a preferred embodiment of the second aspect, the fabrication technique is thermoforming into a mold, such as vacuum forming, and the method comprises the steps of:
providing a thermoplastic sheet,
shaping the thermoplastic sheet into a chamber defined by side walls and a first end wall by thermoforming into a mold, such as applying a vacuum to a mold,
cutting at least one opening in the first end wall,
optionally casting a suspended membrane within the at least one opening.

In a further preferred embodiment, the suspended membrane is cast within the at least one opening.

In a further preferred embodiment, the method according to the second aspect is configured to produce the cell culture insert according to the first aspect.

Further preferably, the cell culture insert according to the first aspect is obtained by the method according to the second aspect.

A third aspect of the disclosure relates to a kit of parts comprising the insert according to the first aspect and optionally a membrane precursor. In a preferred embodiment, the kit of parts comprises the insert according to the first aspect, and optionally a membrane precursor, and/or instructions to form a suspended membrane within the opening of the insert.

A fourth aspect of the disclosure relates to a method of forming a suspended membrane for supporting biomaterials, comprising the steps of:
providing a cell culture insert comprising a chamber defined by at least one side wall and a first end wall, wherein the first end wall has a predefined thickness equal to or below 500 μm and comprises at least one opening,
casting a suspended membrane within the at least one opening.

In a preferred embodiment, the method of forming a suspended membrane includes providing the cell culture insert according to the first aspect and/or the cell culture insert produced according to the second aspect.

Accordingly, a further aspect of the disclosure relates to:

Use of the cell culture insert according to the first aspect for a method of forming a suspended membrane for supporting biomaterials.

Further correspondingly, a further aspect of the disclosure relates to:

A process of casting a suspended membrane for supporting biomaterials using the cell culture insert according to the first aspect.

### DESCRIPTION OF DRAWINGS

The invention will in the following be described in greater detail with reference to the accompanying drawings.

FIG. 1 shows a schematic embodiment of a cell culture insert according to the present disclosure as seen in perspective view: (A) before, and (B) after insertion into a multi well plate. A schematic close-up of the first end wall of the cell culture insert according to the present disclosure as seen in cross sectional view is shown in (C).

FIG. 2 shows a schematic embodiment of a method of producing the cell culture insert according to the present disclosure by vacuum forming.

FIG. 3 shows a schematic embodiment of a method of producing a suspended membrane according to the present disclosure by dispensing of a liquid membrane precursor.

FIG. 4 shows a schematic embodiment of a method of producing a suspended membrane according to the present disclosure by dipping into a liquid membrane precursor.

FIG. 5 shows embodiments of cell culture inserts according to the present disclosure obtained by vacuum forming of PMMA, as seen in side views and bottom views. The height of the chambers is 10 mm (left in FIGS. 5A) and 16 mm (right in FIG. 5A). The relationship between the chamber height (or depth) in mm, and the thickness of the first end wall (bottom thickness) in μm of the chamber is shown in FIG. 5B, as further described in Example 1.

FIG. 6 shows embodiments of cell culture inserts according to the present disclosure obtained by vacuum forming of PET-G, as seen in side views and bottom views. The height of the chambers is 10 mm (left in FIGS. 5A) and 16 mm (right in FIG. 5A). The relationship between the chamber height (or depth) in mm, and the thickness of the first end wall (bottom thickness) in μm of the chamber is shown in FIG. 5B, as further described in Example 2.

FIG. 7 shows cultures of Caco2 cells on membranes with different thicknesses, and in different magnification. The membranes are formed according to the methods described in FIGS. 3 and 4. The membrane precursors are gelatine cross linked with enzymes. The cells are seeded on the membrane and cultured according cell culture practices.

FIG. 8 shows embodiments of cell culture inserts according to the present disclosure obtained by vacuum forming of PS, as seen in side views and bottom views. FIG. 8A shows side views of an embodiment of cell culture inserts where the chamber is not a truncated cone. The two images show the same set of four inserts with a 90 degree difference in rotation. FIG. 8B show an embodiment of cell culture inserts where the openings in the first end wall are square and arranged in a square grid pattern. FIG. 8C show an embodiment of cell culture inserts where the openings in the first end wall are circle segments arranged in a rotational symmetric pattern. The inserts are further described in Example 3.

FIG. 9 shows the measured thickness of gelatine gel in the apertures in the bottom of the wells formed in PET-G, as further described in Example 2. The thickness correlate with the bottom thickness, as further described in Example 6.

### DETAILED DESCRIPTION

The invention is described below with the help of the accompanying figures. It would be appreciated by the people skilled in the art that the same feature or component of the device are referred with the same reference numeral in different figures. A list of the reference numbers can be found at the end of the detailed description section.

### Chamber

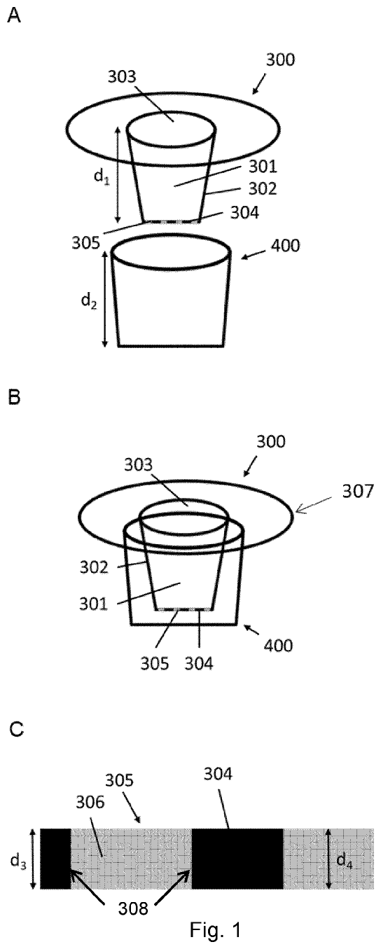

FIG. 1A shows a schematic embodiment of a cell culture insert 300 according to the present disclosure as seen in perspective view. The insert comprises a chamber 301 or a cavity defined by one or more surrounding side walls 302 and a first end wall 304, where the first end wall 304 may form a partially closed end. The chamber's second end wall 303 is placed oppositely to the first end wall 304, and the second end wall 303 may form an open end surrounded by a flange 307, as illustrated in FIG. 1A.

Accordingly, the flange may be suitable for supporting a lid covering the open end of the chamber, or suitable as a hanger flange.

The insert 300 may advantageously be configured dimensionally for at least partially insertion or fitting into a well 400 of e.g. a multi well plate, as sketched in FIG. 1A showing the insert immediately before insertion, and FIG. 1B after insertion. The flange 307 may be configured to abut a top of the well plate, such that the insert is hanging by the flange, i.e. functioning as a hanger flange, as shown in FIG. 1B. This may for example be obtained by the height d₁ of the chamber 301 being smaller than the height d₂ of the well 400. Conventional multi well plates may include 6, 12, 24, 48, or 96 wells, where the height of each well typically is between 11-18 mm, such as 11 or 15-18 mm, and the diameter of each well typically is between 6 -36 mm, such as 6-7 mm, 10-13 mm, 15-18 mm, 22-23 mm, or 34-36 mm.

In an embodiment of the disclosure, the insert and/or the chamber of the insert, are dimensionally configured for insertion into a multi well plate. In a further embodiment, the chamber height is between 2-25 mm, more preferably between 3-20 mm, and most preferably between 4-18 mm, such as 5, 10, 14, or 16 mm. In a further embodiment, the chamber diameter is between 3-50 mm, more preferably between 4-40 mm, and most preferably between 5 -35 mm, such as 6, 7, 10, 13, 15, 18, 20, 22, 23, or 34 mm.

The chamber 301 may comprise cells seeded together with cell culture medium, and the well 400 may likewise contain cell culture medium and/or be populated by cells, thereby supporting cell culture modalities.

Advantageously, the first end wall 304 has a thickness d₃ that is flexibly and reliably controlled, and where the controlled thickness may be predefined and is equal to or below 500 μm, such as between above 100 μm and below 400 μm, e.g. between 110-350 μm or between 110-210 μm. The first end wall further comprise at least one opening 305. For example, the first end wall may comprise one opening or four openings as sketched in FIGS. 1A-B. The precisely controlled and predefined wall thickness facilitates that the opening is configured to comprise a suspended membrane layer, where the thickness may also be flexibly and reliably controlled and predefined, specifically with a controlled thickness below 500 μm, as further described below. The first end wall is advantageously essentially planar, thereby defining a first end wall plane, such that the wall thickness d₃ of the first end wall may be precisely controlled. Accordingly, the first end wall may also be considered as an aperture plane.

The boundaries of the one or more openings, or apertures, are defined by an opening perimeter 308, where the perimeter surface forms the side walls of the opening cavity 306. The opening cavity is thus defined by the upper and lower surface plane of the first end wall 304, and the side walls of the opening perimeter, as sketched in FIG. 1C, and the height of the perimeter surface may correspond to d₃ as sketched in FIG. 1C. The perimeter surface may be configured for supporting a suspended film or membrane by surface tension, including the formation of the suspended film or membrane, meaning that the suspended film is perpendicularly oriented to the perimeter surface. Accordingly, only the lateral edges of the suspended film adhere to, or are supported by, the perimeter surface, and the film or membrane may be suspended, optionally without use of adhesives or glue. However, to further support the stability of the suspended film, adhesives and/or glue may be applied to the perimeter surface.

For example, as indicated in FIG. 1C, the horizontally oriented suspended membrane 306 is supported only at the lateral edges of the membrane by the vertically oriented perimeter surface 308 by surface tension, similar to a soap film suspended on a frame for a soap bubble blower toy. Thus, the suspended membrane may be attached and adhered to the perimeter surface without the use of adhesives.

In an embodiment of the disclosure, the at least one opening is defined by an opening perimeter, the perimeter having a surface configured for supporting a suspended membrane at the edges of the membrane by surface tension. In a further embodiment, the perimeter surface is essentially perpendicular to the first end wall plane. In a further embodiment, the perimeter surface does not comprise adhesives or glue.

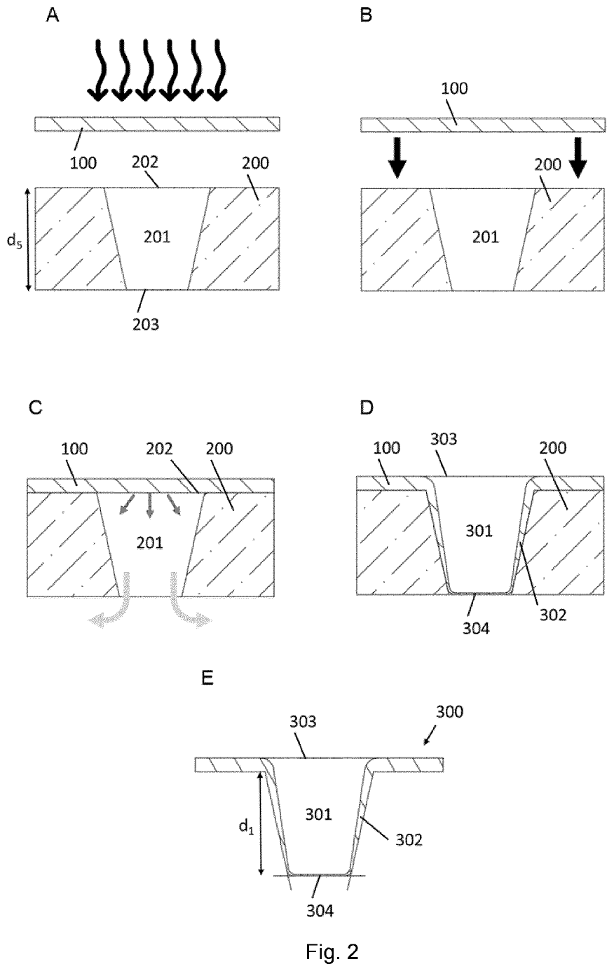

Thus, only the opening perimeter is in contact with the suspended membrane, and the suspended membrane may be supported at the lateral edges of the membrane by surface tension, optionally without any adhesives or glue, thereby facilitating that the thickness of the suspended membrane may be precisely controlled via the dimensions of the opening perimeter. Accordingly, the dimensions of the opening perimeter is determined by the thickness of the first end wall in the vicinity of the opening, and thus the first end wall is advantageously essentially planar with an essentially uniform thickness, such that the height of the perimeter is similar to the thickness of the first end wall, particularly in the vicinity of the opening. The first end wall may be formed by thermoforming, such as vacuum forming, to obtain a precisely controlled and predefined thickness of the first end wall. Thermoforming into a mold may involve slight curvature to the end wall near the mold edges, as indicated in FIG. 2. However, the skilled person will know to disregard such minor edge curvatures, when evaluating the essential planar and uniform thickness of the first end wall, particularly in the vicinity of the opening, where the openings are located at a distance to the mold edges.

Thus, advantageously, the height of the side walls of the opening cavity, corresponding to the height of the perimeter surface, is identical to the wall thickness d₃ of the first end wall, such that the opening cavity may support in situ formation of a suspended film with a precisely controlled thickness d₄, which is essentially equal or similar to the thickness of the first end wall d₃. Hence, the thickness of the first end wall is advantageously uniform and predefined, particularly in the vicinity of the openings. The manufacturing of openings in a sheet such as an end wall, e.g. by cutting, piercing or punching the openings, may result in sheet deformation. For example openings formed by piercing may result in bulging around the opening. Hence, advantageously suitable methods for manufacturing the openings should be chosen such that the deformation of the opening perimeter is avoided or minimized. For example, laser ablation and/or by polishing the bulges subsequent to forming the opening may be used.

In an embodiment of the disclosure, the first end wall is essentially planar with a predefined uniform thickness.

An opening 305 may be configured to comprise a suspended membrane 306, as seen in the cross sectional close-up of the first end wall 304 in FIG. 1C. The stability and geometry of the suspended membrane may depend on various factors including: the wetting properties and surface tension of the interfacing materials, i.e. the membrane-wall-atmosphere interface, the viscosity of the membrane material, the thickness of the wall, and the geometry of the opening. It was surprisingly found that the opening and the perimeter surface dimensions may be particularly suitable for supporting a suspended film or membrane by surface tension, including the formation of the suspended film or membrane, when the film or membrane comprises a hydrogel or consists of a hydrogel. Thus, a particularly precisely controlled and stable hydrogel film with a particularly precisely predefined thickness may be obtained. Accordingly, the hydrogel film may be stably suspended without the use of adhesives, and are advantageously suspended by primarily surface tension forces at the edges of the film. It was further surprisingly found that for a first end wall thickness equal to or below 500 μm, the thickness of the membrane da may be determined and controlled by the thickness d₃ of first end wall, as illustrated in FIG. 1C.

In an embodiment of the disclosure, the cell culture insert comprises a chamber defined by at least one side wall and a first end wall, wherein the first end wall has a thickness equal to or below 500 μm and comprises at least one opening. In a further embodiment, the opening is configured for comprising a suspended membrane.

Accordingly, the suspended membrane may have a flexibly and reliably controlled thickness. Further, the suspended membrane is not deposited on another substrate, e.g. on a Transwell® insert with limited porosity and mass transport.

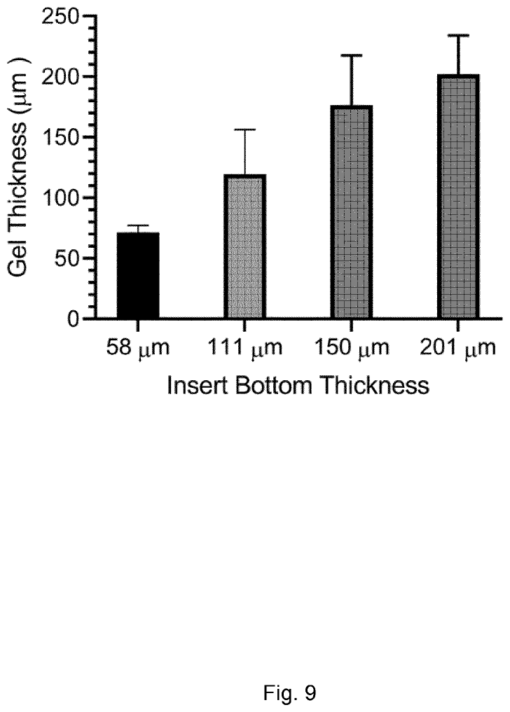

To further improve the mass transport across the membrane, the thickness d₃ of the first end wall 304 of the chamber is advantageously below 500 μm or 300 μm. The thinner the thickness, the higher the possible mass transport through the correspondingly suspended membrane. For example, a molecule diffuses 100 fold quicker through a 20 μm membrane compared to a 200 μm membrane. However, also the thinner the thickness, the more fragile the material of the insert and the corresponding membrane. Suspended membranes may have improved mechanical properties compared to membranes with a corresponding thickness which are casted on a support. To obtain sufficient mass transport across the membrane as well as sufficient mechanical robustness, the thickness of the first end wall may advantageously be at least above 100 μm and below 400 μm, such as between 110-350 μm or between 110-210 μm, as shown in FIG. 9.

In an embodiment of the disclosure, the first end wall has a thickness of between 5-300 μm, more preferably between 10-250 μm, and most preferably between 15-200 μm, such as 20, 50, 70, 90, or 100 μm.

In another embodiment of the disclosure, the first end wall has a thickness which is above 100 μm and below 400 μm, more preferably between 110-350 μm or 110-210 μm.

Openings The stability and geometry of a suspended membrane 306 may also depend on the geometry of the opening 305, in addition to the thickness of the wall d₃ and the wetting properties and viscosity. The geometry of the openings may further affect the efficiency when testing of barrier cell models, and cell culture modalities.

It is found that cell culture inserts with one or more openings 305, and/or openings 305 constituting a higher area fraction of the first end wall 304, may be particularly efficient for testing of cell cultures, and especially barrier cell models, and cell culture modalities. The one or more openings and/or the area fraction of the openings may further support the stability and geometry of a suspended membrane, specifically that the thickness of the membrane da may be controlled by the thickness d₃ of first end wall. The advantageous area fraction of the first end wall may be obtained by a single opening or by multiple openings providing a corresponding area fraction, such as 50 openings, or even hundreds or thousands openings.

In an embodiment of the disclosure, the insert comprises one of more openings, such as 1-50 openings, optionally between 5-40, and more optionally between 10-30, such as 13, 15, 17, 19, 21, 23, 25, 27, or 29. In a further embodiment, the cross sectional area of the openings is between 0.1-100% of the area of the first end wall, more preferably between 1-95%, and most preferably between 10-90%.

It is further found that cell culture inserts with specific shapes and dimensions of the openings 305, and/or specific distribution of the openings within the first end wall 304, may support the stability and geometry of a suspended membrane, specifically that the thickness of the membrane da may be controlled by the thickness d₃ of first end wall.

Advantageously, the openings are approximately circular or oval e.g. with diameters between 10-10.000 μm (corresponding to 10 mm), and/or distributed in a rotational symmetric pattern as seen from an end view. For example, the openings are advantageously placed within the first end wall in an approximately equidistant hexagonal pattern. For example, the distance between the centres of two circular holes may be at or between 1.5-2 mm, resulting in a distance between the edges of the circular holes of less than 1 mm. However, depending on the number and distribution pattern of the openings and the size of the insert end wall, the distance between neighbouring openings may for example be 50 mm for an insert with a few openings in a large insert, or 10 μm or less for an insert with a tight pattern of the openings.

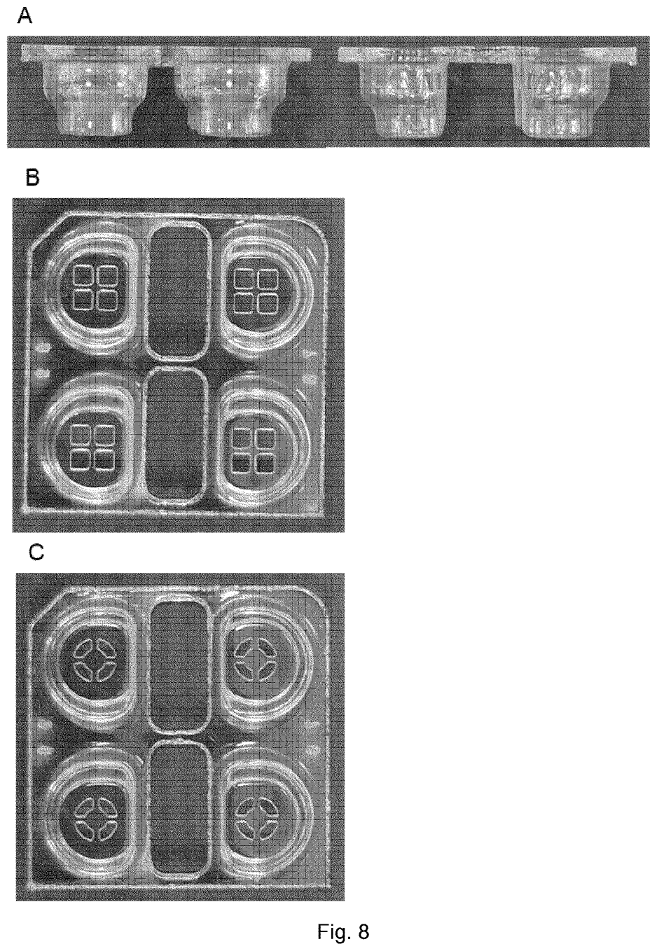

The shape of the openings may further advantageously be polygonal, such as approximately rectangular or squared as seen in cross sectional view, as shown in FIG. 8B. For ellipsoid openings, the smallest diameter may be 10-2000 μm, preferably between 10-1000 μm such as 250, 500, 750 and 1000 μm.

In an embodiment of the disclosure, the shape of the openings in cross sectional view is selected from the group consisting of: approximately circular, oval, ellipsoid, or polygonal such as squared or rectangular. In an embodiment of the disclosure, the openings may be defined by a diameter, where the openings have a diameter of between 10-10.000 μm, more preferably between 50-5.000 μm, and most preferably between 100-1.500 μm, such as 250, 500, 750, or 1.000 μm.

The openings may for example be distributed in a rotational symmetric pattern within the first end wall. For example, the openings may form an approximately equidistant hexagonal pattern, or the openings may describe a circle segment, as seen from the end view in FIG. 8C, or a grid pattern, such as a squared grid pattern, as seen in FIG. 8B.

In a further embodiment, the openings are distributed rotational symmetrically within the first end wall, such as in an approximately equidistant hexagonal pattern. In a further embodiment, the openings describe a circle segment in cross sectional view. In a further embodiment, the openings are distributed in a grid pattern within the first end wall, such as in a square grid pattern.

Examples 4-5 further describe examples of openings according to the present disclosure, where the openings are shaped as squares or segments of a circle.

### Side Wall

The stability and geometry of a suspended membrane 306 may also significantly depend on the general geometry of the chamber 301. This is due to the geometry or thickness of the side walls 302 being related to the thickness of the first end wall 304, which is specifically the case for inserts obtained by thermoforming techniques such as vacuum forming. For example, the dimensions of the side walls may directly determine the thickness of the first end wall, as shown in FIG. 2 and further described below.

The chamber geometry may further affect how precise the thickness of the first end wall 304 may be controlled.

To obtain a first end wall 304 with a thickness d₃ that is flexibly and reliably controlled to a thickness equal to or below 500 μm, the side walls 302 are advantageously partially tapered or sloped. For example, the tapering may extend along the entire length of the side walls as illustrated in FIGS. 2D-E, or extend along a part of the length. The tapering angle may further be constant along the length of the side walls as shown in FIGS. 2D-E, or vary along the length. Optionally, the tapering angle is at least 1 degree along the entire length. Optionally, the tapering is combined with the bulk geometry of the insert also being tapered, i.e. having the shape of a truncated cone as shown in FIG. 2.

In an embodiment of the disclosure, the thickness of the side walls is at least partially tapered. In a further embodiment, the tapering angle relative to a vertical side wall is between 1-45 degrees, more preferably between 2-20 degrees, and most preferably between 3-10 degrees, such as 3, 5, or 7 degrees.

Advantageously, the tapering is constant towards the first end wall 304 and extends along the entire length of the side wall, such that thickness of the first end wall 304 may be more precisely controlled. From FIG. 2 it follows that the side wall thickness will be thinner than the starting material sheet thickness, and that the thickness decreases as the material is stretched/thinned and the height of the insert increases. The decrease will also depend on the aspect ratio between the top diameter and height, since this affects the thinning. For example, the side wall thickness may be between 1-1.5 mm at the top of the insert, and much thinner at the bottom near the first end wall. The thickness of the tapered side walls 302 is advantageously between 0.01-5 mm over the entire length to facilitate a first end wall thickness equal to or below 500 μm.

In an embodiment of the disclosure, the thickness of the side walls is tapered towards the first end wall. In a further embodiment, the thickness of the side walls along the entire length is between 0.01-5 mm, more preferably between 0.03-3 mm, or more preferably between 0.05-2.

Advantageously, the chamber is manufactured by a shaping technique which allows simultaneous control of the thickness of the side walls 302 and the first end wall 304.

For example, the chamber may be manufactured by a molding process, where the thickness of the first end wall 304 is dependent on the height of the mold d₅, because the amount of material available for the side walls and end wall is constant. For example, FIG. 2 shows an embodiment of a vacuum forming process, where the height of the mold determines the thickness of the first end wall.

It is found that the thickness of the first end wall may be particularly precisely and efficiently controlled by a shaping technique, and especially a molding process, if the chamber 301 and/or the side walls 302 are shaped as a cylinder or a hollow truncated cone. For a hollow truncated cone, the diameter of the first end wall 304 of the chamber is smaller than the diameter of the second end wall 303. Similar control may be obtained by other shapes, optionally where only a part of the chamber forms a truncated cone, as shown in FIG. 8A.

In an embodiment of the disclosure, at least a portion of the side walls form a cylinder or a hollow truncated cone.

The first end wall may be planar as shown in e.g. FIG. 1 or dome shaped, such as shaped as a half sphere.

In one embodiment of the disclosure, the first end wall is planar or domed shaped, such as shaped as a half sphere.

### Chamber Manufacture

The cell culture insert may be obtained by any shaping technique, where the chamber geometry and specifically the thin thickness of the first end wall may be precisely and flexibly controlled. It is found that the thickness may flexibly and precisely controlled by shaping techniques such as injection molding, 3D printing, casting, and thermoforming. Preferably, the chamber is manufactured by thermoforming techniques such as vacuum forming where specifically thin and controlled thicknesses may be obtained.

In an embodiment of the disclosure, the insert is obtained by a shaping technique selected from the group consisting of: injection molding, 3D printing, casting, and thermoforming, optionally into a mold, such as vacuum forming, and preferably obtained by vacuum forming.

Chambers with flexibly and precisely controlled wall thickness are further advantageously based on polymeric materials, which also provides for the opening being formed by most cutting techniques. Examples of suitable polymeric materials include: polymethylmethacrylate (PMMA), polyethylene terephthalate glycol (PET-G), polyvinyl chloride, polycarbonate, polysulfone, polystyrene (PS), polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and any combinations thereof. In an embodiment of the disclosure, the insert comprises a polymeric material.

Optionally the thermoplastic material is selected from the group consisting of: polymethylmethacrylate (PMMA), polyethylene terephthalate glycol (PET-G), polyvinyl chloride, polycarbonate, polysulfone, polystyrene (PS), polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and any combinations thereof, and preferably selected from the group consisting of: PS, PMMA, PET such as PET-G, and combinations thereof.

To ensure that the casted membrane is stably suspended, and that the suspended membrane may be supported at the lateral edges of the membrane by surface tension, optionally without any adhesives or glue, and thereby facilitating that the thickness of the suspended membrane may be precisely controlled via the dimensions of the opening perimeter, the insert and the first end wall advantageously comprises a hydrophilic thermoplastic material, such as PS, PMMA, and combinations thereof, and advantageously does not comprise hydrophobic materials, such as polyethylene and/or polypropylene.

In an embodiment of the disclosure, the first end wall comprises a hydrophilic material, such as polystyrene. In a further embodiment, the first end wall consists of polystyrene.

The polymeric materials facilitate that the openings 305 may be formed by most cutting techniques and with flexible shapes. For example, the shape of the openings may be circular, oval, ellipsoid, polygonal, rectangular, or quadrilaterals. Accordingly, the dimension of the opening may be characterized by a diameter, length-width ratio, and diagonals of squares or rectangles. The openings are most precisely and efficiently produced using methods such as drilling, milling, laser ablation, water cutting and chemical etching. However, the manufacturing of openings in a sheet by cutting, piercing or punching, may result in sheet deformation, particularly bulging around the opening edges. Hence, advantageously suitable methods for manufacturing the openings should be chosen such that the deformation of the opening perimeter is avoided or minimized. For example, laser ablation and/or by polishing the bulges subsequent to forming the opening may be used.

In an embodiment of the disclosure, the openings are obtained by a cutting process selected from the group consisting of: laser ablation, drilling, milling, punching, chemical etching, and combinations thereof, optionally obtained by CO₂ laser ablation or UV laser ablation. In a further embodiment, the openings are manufactured such that the first end wall is essentially planar at the edges of the openings.

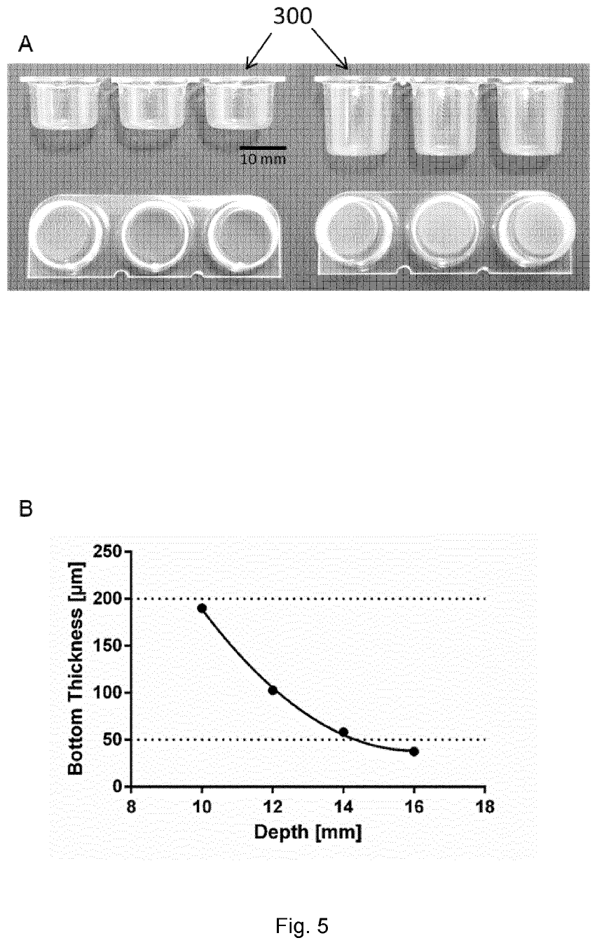

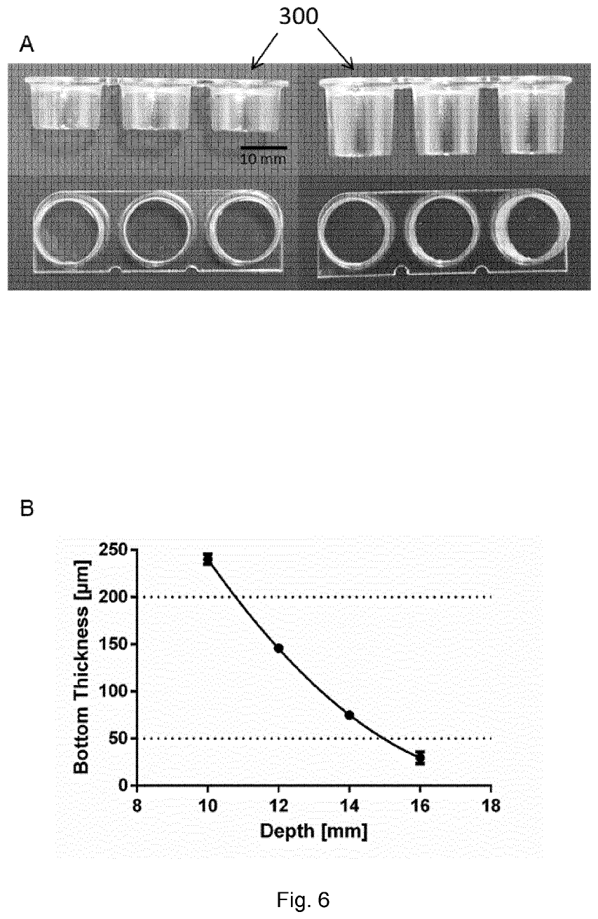

It is found that chambers with flexibly and precisely controlled wall thickness are advantageously produced by vacuum forming, as illustrated in FIG. 2 and further described in Examples 1-2 and related FIGS. 5-6.

FIG. 2A shows a material sheet 100 being heated to a temperature suitable for forming or shaping, which for a polymeric material typically means a temperature above the glass transition temperature of the material. The material sheet 100 is suspended above a mold 200 with one or more cavities 201, with a height d₅, an open first end 202, and a closed second end 203. Optionally, the mold may also be heated, and the mold may comprise venting holes to facilitate the forming (not shown in the Figure).

FIG. 2B shows the material sheet 100 being put into contact with the mold 200 such that the one or more cavities 201 are covered.

FIG. 2C shows air being evacuated from the one or more cavities 201 through the venting holes, as indicated by the light grey arrows. The positions of the venting holes may be anywhere within the cavity, such that they may facilitate air evacuation. The evacuation of air contained in the cavity 201 leads to the part of the material sheet 100 in contact with the open first end 202 of cavity 201 being pulled down into the cavity 201.

FIG. 2D shows the material sheet 100 being stretched to take on the shape of the cavity 201. Hence, the planar sheet is shaped into a corresponding chamber defining a secondary cavity 301 with an open end 303, a closed end 304, and one or more sidewalls 302. Optionally, the mold 200 may include a cavity 201 that is shaped as a truncated cone, as shown in FIG. 2. Accordingly, the planar sheet will be shaped into a chamber having the shape of a truncated cone.

FIG. 2E shows the insert 300 after forming and removal from the mold. The chamber 301 is defined by the side walls 302 which are tapered, with decreasing thickness towards the closed end first end wall 304, along the height d₁, due to the forming process. The process inherently implies that the thickness of the side walls are less than the thickness of the material sheet 100.

The thickness of the closed first end wall 304 is likewise less than the thickness of the material sheet 100 and may be precisely controlled by the shaping process parameters. For example, it follows that the thickness of the first end wall may depend on, but not limited to, the factors: the mold cavity depth d₅, the area of the mold open first end 202, the area of the mold second end 203, the thickness and material of the material sheet 100, and the forming temperature.

It is found that precise and flexible control of the thickness of the first end wall 304, when the thickness is equal to or below 500 μm, may be advantageously obtained based on sheets with a thickness between 0.5-5 mm. It is further found advantageous that the vacuum forming, or other thermoforming process, is based on a female mold, and/or carried out at a temperature above the material glass transition temperature of the respective materials used. Accordingly, for some materials, the process may be carried out at between 100-200 °C. For example, the glass transition temperature is respectively 113 °C and 70 °C for Plexiglass 99524 and PET-G. However thermoplastic materials with significantly higher or lower glass transition exist.

In an embodiment of the disclosure, the sheet has a thickness of between 0.1-5 mm or 0.5-5 mm, such as 1.0, 1.5, or 2.0 mm, and preferably is above 1.0 mm. In a further embodiment, the vacuum forming is based on a female mold. In a further embodiment, the vacuum forming is carried out at a temperature above the glass transition of the insert material.

Examples 1-3 further describe examples of inserts according to the present disclosure, where the insert comprises and/or consists of PMMA, PET-G, or PS.

### Membrane and Method of Forming Membrane

The cell culture insert 300 is configured for comprising a suspended membrane 306 within an opening 305, as e.g. shown in the cross sectional close-up in FIG. 1C, where the opening comprises the suspended membrane within the opening cavity 306, where the suspended membrane is supported at the lateral edges of the membrane by surface tension. A kit of parts may comprise the cell culture insert without the membrane, but together with parts for producing the suspended membrane on demand. Accordingly, when the membrane is produced within the opening, the cell culture insert comprises a suspended membrane.

In an embodiment of the disclosure, the opening comprises a suspended membrane, preferably within the opening cavity. In a further embodiment, the suspended membrane is supported at the edges of the membrane.

Advantageously, the suspended membrane 306 comprises a membrane precursor 500, which facilitates a stable suspended membrane and a controlled membrane thickness and porosity. Hence, advantageously, the membrane precursor comprises sufficient wetting properties and viscosity relative to the geometry and thickness of the opening 305.

The porous membrane 306 may be formed from any suitable material since the wetting properties and viscosity may be controlled by additives and/or surfactants. For porous membranes intended to be used for cell culture and to contact cells or culture media, the membrane advantageously is compatible with the cells and the media. Hydrogels may be particularly suitable for being populated by cells. Examples of hydrogels include: gelatin, collagen, Matrigel®, alginate, agarose, fibrin gels, peptide gels, and any combinations thereof. For example, the cells may be added to the liquid precursor that subsequently form the porous membrane. Further, hydrogels may be tailored to a specific porosity. For example, hydrogels may comprise a porosity above 5% and a pore distribution which is similar to real 3D tissue and remodelled tissue with improved cell movement capacity.

In a further embodiment, the membrane comprises a hydrogel. Optionally, the hydrogel is selected from the group consisting of: gelatin, collagen, Matrigel®, alginate, agarose, fibrin gels, peptide gels, and any combinations thereof. In a further embodiment, the membrane has a porosity of above 40%, preferably between 50-99%, such as 70, 80, 85, or 95%.

The membrane precursor, such as a hydrogel, may also be configured to have sufficient wetting properties and a viscosity relative to the geometry and thickness of the opening, such that the stability and geometry of the suspended membrane may be improved. This may e.g. be obtained by additives and/or surfactants. Specifically, the membrane precursor may be configured such that the thickness of the suspended membrane da may be determined and controlled by the thickness d₃ of first end wall, as illustrated in FIG. 1C and further described in Example 6.

In an embodiment of the disclosure, the thickness of the membrane is essentially similar to the thickness of the first end wall, such as equal to or below 500 μm, more preferably equal to or below 200 μm, and most preferably equal to or below 100 μm, and/or the thickness of the membrane is essentially similar to the thickness of the first end wall, such as above 100 μm and below 400 μm, more preferably between 110-350 μm or between 110-210 μm.

The suspended membrane 306 with precisely and flexibly controlled thickness and porosity may be obtained by casting a liquid membrane precursor 500 into the opening 305. The wetting properties and viscosity of a liquid precursor may be controlled by the liquid composition, such the casted suspended membrane may be controlled relative to the geometry and thickness of the opening. Specifically, it is found that the thickness of the casted liquid membrane da may be determined and controlled by the thickness d₃of first end wall, as illustrated in FIG. 1C. Preferably the liquid membrane precursor is a hydrogel precursor, such as a gelatine solution.

In an embodiment of the disclosure, the suspended membrane is casted based on a liquid membrane precursor, optionally a hydrogel precursor, such as a gelatine solution.

The stability and geometry of the suspended membrane 306 may also depend on the casting method. Advantageously, the membrane or hydrogel is casted by contacting the opening with a liquid membrane precursor via dispensing the liquid into the opening, and/or by dipping the opening into the liquid.

In an embodiment of the disclosure, the suspended membrane is formed by contacting at least one opening with a liquid membrane precursor via dispensing the liquid, and/or by dipping into the liquid.

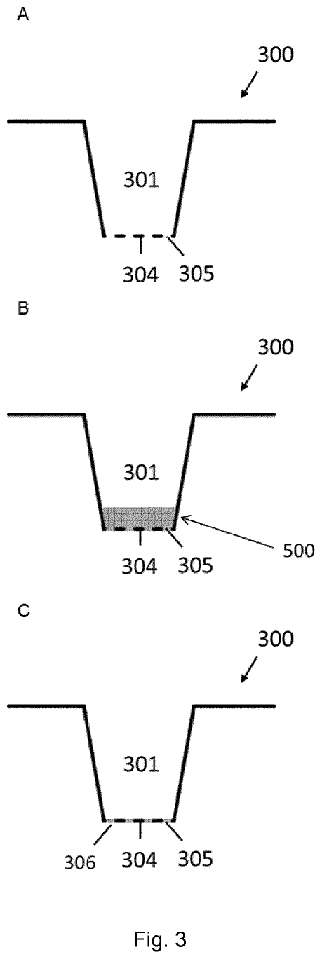

FIG. 3 shows a schematic embodiment of a suspended membrane 306 casted or deposited within the openings 305, which is made by dispensing of a liquid membrane precursor 500 into the chamber 301 comprising the opening 305.

FIG. 3A shows an insert 300 with a number of openings 305 in the first end wall 304. A suitable amount of liquid membrane precursor 500 is dispensed, e.g. pipetted into the chamber 301, as shown in FIG. 3B. It follows that the size of the openings 305 must be chosen such that surface tension can prevent the liquid precursor from exiting the insert through the openings 305.

FIG. 3C the insert after the liquid precursor has been withdrawn. Due to the wetting properties relative to the opening, and the physical properties of the liquid precursor, e.g. the viscosity, the liquid precursor remains within the openings 305 and will fill out the cavity of the opening. Thus, the casted membrane has a thickness essentially similar to the thickness of the first end wall. Accordingly, it is possible to control the membrane thickness.

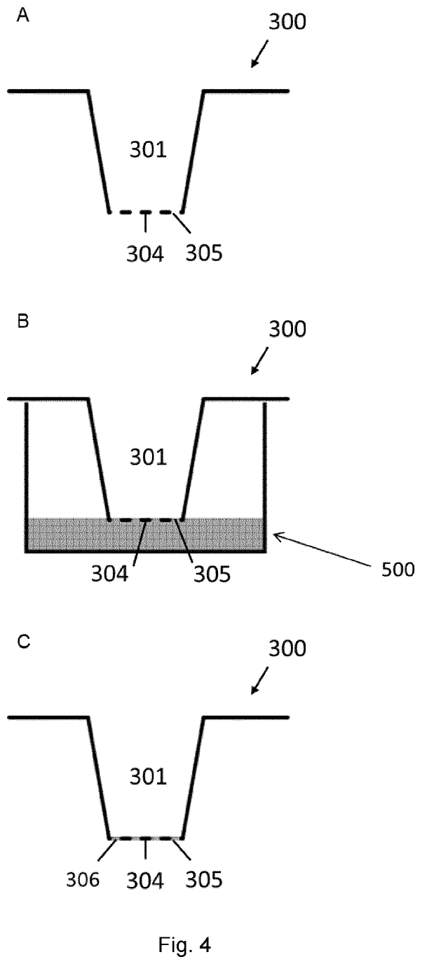

FIG. 4 shows a schematic embodiment of a suspended membrane 306 casted or deposited within the openings 305, which is made by dipping an opening into a liquid membrane precursor 500.

FIG. 4A shows an insert 300 with a number of openings 305 in the first end wall 304. The insert is lowered into a container to contact the openings with the surface of a liquid membrane precursor 500, as shown in FIG. 4B. It follows that the amount of liquid precursor is chosen such that the openings just touch the surface of the liquid precursor. Due to the wetting properties and surface tension relative to the opening, and the physical properties of the liquid precursor, e.g. the viscosity, the liquid precursor will enter the openings 305.

FIG. 4C shows the same insert as in 4B after the insert has been lifted out of the container and out of contact with the surface of the precursor. Due to the wetting properties and surface tension relative to the opening, and the physical properties of the liquid precursor, e.g. the viscosity, the liquid precursor will fill out the cavity of the opening. Thus, the casted membrane has a thickness essentially similar to the thickness of the first end wall. Accordingly, it is possible to control the membrane thickness.

The membrane precursor 500 can be converted using a variety of known methods into a hydrogel 306 (FIG. 4C). Examples are light-, heat-, ionic-, enzymatic- or protein protein interactions based-cross linking of precursors 500. It is surprisingly found that the height of the hydrogels 306 in the apertures 305 is determined by the thickness of the bottom 304, as shown in FIG. 9 and further described in Example 6.

Advantageously, the membrane is formed by using the cell culture insert or the chamber according to the present disclosure comprising an opening, and the casting is carried out within the opening perimeter, such that casted and formed suspended membrane is supported at the opening perimeter by surface tension. Accordingly, the thickness of the suspended membrane may be precisely controlled and predefined, since it may be essentially similar to the predefined thickness of the first end wall.

Hence, an aspect of the disclosure relates to a method of forming a suspended membrane for supporting biomaterials, comprising the steps of:
providing a cell culture insert comprising a chamber defined by at least one side wall and a first end wall, wherein the first end wall has a predefined thickness equal to or below 500 μm and comprises at least one opening,
casting a suspended membrane within the at least one opening, where the at least one opening is defined by an opening perimeter, and the casting is carried out within the opening perimeter, such that formed suspended membrane is supported at the opening perimeter by surface tension.

The casting process and the properties of the casted and formed suspended membrane will depend on the precursor used for the casting and the casting method. Advantageously, the casting is based on a liquid membrane precursor such as a gelatine solution, and the casting is obtained by contacting the opening with the precursor either by dispensing and/or dipping.

In an embodiment of the disclosure, the casting is based on a liquid membrane precursor, optionally a hydrogel precursor, such as a gelatine solution. In a further embodiment, the suspended membrane is formed by contacting the at least one opening with a liquid membrane precursor via dispensing the liquid, and/or by dipping into the liquid.

The casting process facilitates that only the opening perimeter is in contact with the suspended membrane, and the suspended membrane may be supported at the lateral edges of the membrane by surface tension, optionally without any adhesives or glue.

This further means that the thickness of the suspended membrane may be precisely controlled via the dimensions of the opening perimeter, and particularly via the thickness of the first end wall. Advantageously, the first end wall is essentially planar with a uniform thickness, particularly in the vicinity of the opening, such that the thickness of the suspended membrane may be precisely controlled to thickness enabling sufficient mass transport across the membrane as well as sufficient mechanical robustness.

In an embodiment of the disclosure, the thickness of the suspended membrane is essentially similar to the predefined thickness of the first end wall, such as equal to or below 500 μm, more preferably equal to or below 200 μm, and most preferably equal to or below 100 μm, or such as above 100 μm and below 400 μm, more preferably between 110-350 μm or between 110-210 μm.

The suspended membranes are supported at the opening perimeter and the lateral edges of the membrane, and thus particularly suitable for supporting biomaterials including a culture of cells.

In an embodiment of the disclosure, the membrane is configured to support a culture of cells seeded onto the membrane, and/or supports culture of cells seeded into the membrane precursor. In a further embodiment, the cells are selected from the group consisting of: cancer cell lines, primary cell lines, stem cells, differentiated stem cells to a variety of cell types, tissue slices from biopsies, and combinations thereof.

### Cell Culture Using Inserts With Membranes

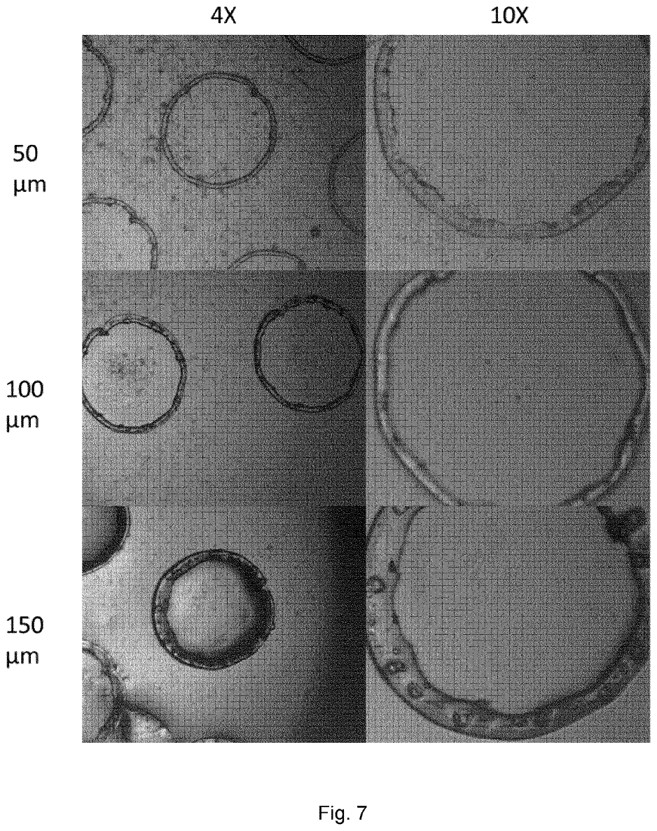

The cells can come from a variety of sources such as cancer cell lines, primary cell lines from a variety of sources and stem cells, including stem cells from a variety of cell types. For example, FIG. 7 shows cultures of Caco2 cells on membranes with different thicknesses, and in different magnification, where the membrane precursors are gelatine cross linked with enzymes. The cells are seeded on the membrane and cultured according cell culture practices.

In another embodiment, tissue slices from biopsies are cultured on the membrane. Cells are either plated on the membrane 306 or cased inside the membrane 306. Cells can be plated on either side of the membrane 306 or any combination of cased inside and plating on the polymerised membrane 306 surface.

In an embodiment of the disclosure, the membrane supports culture of cells seeded onto the membrane, and/or supports culture of cells seeded into the membrane precursor.

In an embodiment of the disclosure, the cells are selected from the group consisting of: cancer cell lines, primary cell lines, stem cells, differentiated stem cells to a variety of cell types, tissue slices from biopsies, and combinations thereof.

Examples 7-10 further describe examples of use of the inserts according to the present disclosure for cell culturing.

### EXAMPLES

The invention is further described by the examples provided below.

### Example 1—Manufacture of a Cell Culture Insert Comprising PMMA

Cell culture inserts according to the present disclosure were manufactured by thermoforming a polymeric material, and specifically by a vacuum forming, as illustrated in FIG. 2.

In an example, the polymeric material is PMMA, and a sheet of 1 mm PMMA (specifically Plexiglas® 99524) was used as starting sheet 100.

The used mold 200 was one of four molds with six identical mold cavities 201, respectively. The mold cavities were shaped as truncated cones with tapering side walls having a first open end 202, and a second closed end 203, as shown in FIG. 2A. The open ends were identical in diameters, but the mold cavities had different height d₅ or depths. Four different mold heights were used having depths of 10, 12, 14, and 16 mm respectively.

The PMMA sheet was mounted in a vacuum forming machine and heated to 170 °C using the machines heating elements. During the process, the desired mold was also placed in the machine. After reaching the desired temperature, the sheet was lowered to contact the mold and creating a vacuum seal against the machine, as illustrated in FIGS. 2B-C. As the air was removed from inside the mold, the PMMA sheet was drawn into the mold to form the desired chamber 301 of the insert, as illustrated in FIG. 2D.

The insert chambers 301 were subsequently cooled down and removed from the mold, as illustrated in FIG. 2E. The manufactured insert has a chamber height d₁, and the insert may be cut into strips comprising 3 chamber, as illustrated in FIGS. 5A and 6A.

FIG. 5A shows the produced chambers in side views (upper image) and bottom views (lower image), i.e. as seen from the first end wall. The height of the chambers is 10 mm (left in FIGS. 5A) and 16 mm (right in FIG. 5A).

FIG. 5B shows the relationship between the chamber height d₅ (or depth) in mm, and the thickness of the first end wall 304 (bottom thickness) in μm of the chamber. It is seen that the mold height and the bottom thickness may be mathematical related as a regression fitted line with n=6.

Hence it follows that by varying the mold height, the thickness of the first end wall may be controlled to a well-defined thickness between ca. 50-200 μm, highlighted by the dotted lines in FIG. 5B.

One or more openings 305 are subsequently cut into the first end wall 304, e.g. by laser ablation or drilling. Optionally, the one or more openings are contacted with a liquid membrane precursor 500 as further described in Example 6, for casting a suspended membrane within the opening, as illustrated in FIG. 1.

### Example 2—Manufacture of a Cell Culture Insert Comprising PET-G

Cell culture inserts comprising different polymeric materials may be produced according to a similar method as described in Example 1. In another example, the polymeric material is PET-G, and a sheet of 1.5 mm PET-G was used as starting sheet 100.

Similar molds as for Example 1 was used. Hence, the used mold 200 was one of four molds with six identical mold cavities 201, respectively. The mold cavities were shaped as truncated cones with tapering side walls having a first open end 202, and a second closed end 203, as shown in FIG. 2A. The open ends were identical in diameters, but the mold cavities had different height d₅ or depths. Four different mold heights were used having depths of 10, 12, 14, and 16 mm respectively.

A vacuum forming process similar to Example 1 and FIG. 2 was applied, except due to the different polymeric material, the process was carried out at 150 °C instead of 170 °C

FIG. 6A shows the produced chambers in side views (upper image) and bottom views (lower image), i.e. as seen from the first end wall. The height of the chambers is 10 mm (left in FIGS. 6A) and 16 mm (right in FIG. 6A).

FIG. 6B shows the relationship between the chamber height d₅ (or depth) in mm, and the thickness of the first end wall 304 (bottom thickness) in μm of the chamber. It is seen that the mold height and the bottom thickness may be mathematical related as a regression fitted line with n=6.

Hence it follows that by varying the mold height, the thickness of the first end wall may be controlled to a well-defined thickness between ca. 25-250 μm.

One or more openings 305 are subsequently cut into the first end wall 304, e.g. by laser ablation or drilling. Optionally, the one or more openings are contacted with a liquid membrane precursor 500 as further described in Example 6, for casting a suspended membrane within the opening, as illustrated in FIG. 1.

### Example 3—Manufacture of a Non-symmetrical Cell Culture Insert Comprising PS

Cell culture inserts comprising different shapes may be produced according to a similar method as described in Example 1. In another example, the polymeric material is PS, and a sheet of 2.0 mm PS was used as starting sheet 100.

Similar molds 200 as for Example 1 was used, but with eight cavities 201 of a different shape. The mold cavities were shaped as truncated cones with a step-in halfway down the height d₅ as well as one essentially flat side wall.

A vacuum forming process similar to Example 1 and FIG. 2 was applied, except due to the different polymeric material, the process was carried out at 180 °C instead of 170 °C

FIG. 8A shows the produced chambers in side views from two different angles 90 degrees apart.

### Example 4—Manufacture of a Cell Culture Insert Comprising Square Openings

Cell culture insert comprising openings 305 with different shapes may be produced according to a similar method as described in Example 1. In another example the one or more openings 305 are cut into the first end wall 304, e.g. by laser ablation or milling, in the shape of a square. FIG. 8B shows square openings 305, cut in a square grid pattern, into the first end wall 304, of inserts produced through the method described in example 3, using laser ablation.

### Example 5—Manufacture of a Cell Culture Insert Comprising Openings in the Shape of Circle Segments

Cell culture insert comprising openings 305 with different shapes may be produced according to a similar method as described in Example 1. In another example the one or more openings 305 are cut into the first end wall 304, e.g. by laser ablation or milling, in the shape of a circle segments. FIG. 8C shows openings 305 in the shape of circle segments, cut in a rotationally symmetric pattern, into the first end wall 304, of inserts produced through the method described in example 3, using laser ablation.

### Example 6—Manufacture of a Cell Culture Insert Comprising a Suspended Membrane

The one or more openings of the produced cell culture inserts of Examples 1-2 are contacted with a liquid membrane precursor 500 for casting a suspended membrane within the opening, as illustrated in FIG. 1.

The contact with the liquid membrane precursor 500 may be obtained by dispensing of the liquid into the opening 305 as illustrated in FIG. 3B, or by dipping the opening into the liquid as illustrated in FIG. 4B. For example, the liquid may be dispensed into the opening by pipetting a predetermined volume of liquid into the chamber 301 and then removing excess liquid.

The liquid membrane precursor may be an aqueous solution of 15 wt % gelatine with a temperature of 37 °C

For the cell culture inserts comprising PMMA or PET-G or PS of Examples 1-3, a mechanical stable and suspended membrane 306 having a membrane thickness d₄essentially the same as the thickness of the first end wall d₃ may be manufactured.

Specifically, suspended membranes with thicknesses equal to or below 500 μm, equal to or below 200 μm, or equal to or below 100 μm, or equal to or below 50 μm may be obtained. This is for example seen in FIG. 9 showing the thickness of the suspended gel as a function of the wall thickness of the first end wall (the insert bottom) of a PET-G, as described in Example 2.

Further, the suspended membranes 306 may have a particularly high and flexible porosity similar to real 3D tissue and modelled tissue with improved cell movement capacity, and particularly suitable for cell culture modalities. For example membrane porosity above 50%, such as between 50-99%, may be manufactured.

### Example 7—Use of a Cell Culture Insert With Membrane in Cell Culture

The suspended membrane 306 produced as described in Example 6 can be used to support cell culture. In one embodiment, Caco2 cells are cultured on the surface of the membrane (FIG. 7). In this example, the precursor is gelatine at 37 degree Celcius and added to chamber 301. The gelatine is cross linked using enzymes. After solidifying in the membrane 306, cells are seeded and cultured using know techniques.

### Example 8. Culturing Inside the Hydrogel

HepG2 cell are mixed with the precursor 500 and casted using the method described in Example 6. The precursor cell mixture is crosslinked and thereby forms a solid hydrogels 306 with cells inside.

### Example 9 Culturing on Top and Bottom Side of Hydrogel

HUVEC and Caco2 cells respectively are cocultured on either side of the membrane 306, using the method previously described in Example 7.

### Example 10 Culturing on Top and Bottom and Inside the Hydrogels

Caco2 cells are co cultured with HepG2 cells and HUVEC cells using a combination of the casting procedure described in Example 6 and the seeding procedure described in Example 8 or 9.

### REFERENCE NUMBERS

100—Sheet
200—Mold
201—Mold cavity
202—Mold first end
203—Mold second end
300—Insert
301—Chamber
302—Side wall
303—Second end wall
304—First end wall
305—Opening
306—Membrane or opening cavity
307—Flange
308—Opening perimeter
400—Well
500—Liquid precursor
d₁—Height of chamber
d₂—Height of well
d₃—Thickness of first end wall
d₄—Thickness of membrane
d₅—Height of mold

### ITEMS

The presently disclosed may be described in further detail with reference to the following items.
1. A cell culture insert comprising a chamber defined by at least one side wall and a first end wall, wherein the first end wall has a thickness equal to or below 500 μm and comprises at least one opening.
2. The insert according to item 1, wherein the at least one opening is defined by an opening perimeter, the perimeter having a surface configured for supporting a suspended membrane at the edges of the membrane by surface tension.
3. The insert according to item 2, wherein the perimeter surface is essentially perpendicular to the first end wall plane.
4. The insert according to any of items 2-3, wherein the perimeter surface does not comprise adhesives or glue.
5. The insert according to any of the preceding items, wherein the first end wall is essentially planar with a predefined uniform thickness.
6. The insert according to any of items 2-5, wherein the first end wall is essentially planar at the edges of the openings, such that the height of the perimeter surface is similar to the predefined uniform thickness of the first end wall.
7. The insert according to any of the preceding items, wherein the first end wall comprises or consists of a hydrophilic material, such as polystyrene.
8. The insert according to any of the preceding items, wherein the first end wall has a thickness of between 5-300 μm, more preferably between 10-250 μm, and most preferably between 15-200 μm, such as 20, 50, 70, 90, or 100 μm.
9. The insert according to any of the preceding items, wherein the first end wall has a thickness which is above 100 μm and below 400 μm, more preferably between 110-350 μm or between 110-210 μm.
10. The insert according to any of the preceding items, comprising one or more openings, such as between 1-50 openings, optionally between 5-40, and more optionally between 10-30, such as 13, 15, 17, 19, 21, 23, 25, 27, or 29.
11. The insert according to any of the preceding items, wherein the cross sectional area of the openings is between 0.1-100% of the area of the first end wall, more preferably between 1-95%, and most preferably between 10-90%.
12. The insert according to any of the preceding items, wherein the shape of the openings in cross sectional view is selected from the group consisting of: approximately circular, oval, ellipsoid, or polygonal such as squared or rectangular.
13. The insert according to item 12, wherein the openings have a diameter of between 10-10.000 μm, more preferably between 50-5.000 μm, and most preferably between 100-1.500 μm, such as 250, 500, 750, or 1.000 μm.
14. The insert according to any of preceding items, wherein the openings are distributed rotational symmetrically within the first end wall, such as in a approximately equidistant hexagonal pattern.
15. The insert according to any of the preceding items, wherein the openings describe a circle segment in cross sectional view.
16. The insert according to any of preceding items, wherein the openings are distributed in a grid pattern within the first end wall, such as in a square grid pattern.
17. The insert according to any of the preceding items, wherein the thickness of the side walls is at least partially tapered.
18. The insert according to item 17, wherein the tapering angle relative to a vertical side wall is between 1-45 degrees, more preferably between 2-20 degrees, and most preferably between 3-10 degrees, such as 3, 5, or 7 degrees.
19. The insert according to any of items 17-18, wherein the thickness of the side walls is tapered towards the first end wall.
20. The insert according to any of items 17-19, wherein the thickness along the entire side wall is between 0.01-5 mm, more preferably between 0.03-3 mm, or most preferably 0.05-2 mm
21. The insert according to any of the preceding items, wherein at least a portion of the side walls form a cylinder or a hollow truncated cone.
22. The insert according to any of the preceding items, wherein the first end wall is planar or domed shaped, such as shaped as a half sphere.
23. The insert according to any of the preceding items, dimensionally configured for insertion into a multi well plate.
24. The insert according to item 23, wherein the chamber height is between 2-25 mm, more preferably between 3-20 mm, and most preferably between 4-18 mm, such as 5, 10, 14, or 16 mm.
25. The insert according to any of items 23-24, wherein the chamber diameter is between 3-50 mm, more preferably between 4-40 mm, and most preferably between 5 -35 mm, such as 6, 7, 10, 13, 15, 18, 20, 22, 23, or 34 mm.
26. The insert according to any of the preceding items, wherein the opening is configured for comprising a suspended membrane, preferably within an opening cavity.
27. The insert according to any of the preceding items, wherein the opening comprises a suspended membrane.
28. The insert according to any of items 26-27, wherein the suspended membrane is supported at the edges of the membrane.
29. The insert according to any of items 26-28, wherein the membrane comprises a hydrogel, optionally selected from the group consisting of: gelatin, collagen, Matrigel®, alginate, agarose, fibrin gels, peptide gels, and any combinations thereof.
30. The insert according to any of items 26-29, wherein the membrane has a porosity of above 40%, preferably between 50-99%, such as 70, 80, 85, or 95%.
31. The insert according to any of items 26-30, wherein the thickness of the membrane is essentially similar to the thickness of the first end wall, such as equal to or below 500 μm, more preferably equal to or below 200 μm, and most preferably equal to or below 100 μm.
32. The insert according to any of items 26-31, wherein the thickness of the membrane is essentially similar to the thickness of the first end wall, such as above 100 μm and below 400 μm, more preferably between 110-350 μm or between 110-210 μm.
33. The insert according to any of items 26-32 wherein the membrane supports culture of cells seeded onto the membrane, and/or supports culture of cells seeded into the membrane precursor.
34. The insert according to item 32 where the cells are selected from the group consisting of: cancer cell lines, primary cell lines, stem cells, differentiated stem cells to a variety of cell types, tissue slices from biopsies, and combinations thereof.
35. The insert according to any of the preceding items obtained by a shaping technique selected from the group consisting of: injection molding, 3D printing,
casting, and thermoforming, such as vacuum forming, and preferably obtained by vacuum forming.
36. The insert according to any of the preceding items comprising a polymeric material, optionally a thermoplastic material selected from the group consisting of: polymethylmethacrylate (PMMA), polyethylene terephthalate glycol (PET-G), polyvinyl chloride, polycarbonate, polysulfone, polystyrene (PS), polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and any combinations thereof, and preferably selected from the group consisting of: PS, PMMA, PET such as PET-G, and combinations thereof.
37. The insert according to any of the preceding items wherein the openings are obtained by a cutting process selected from the group consisting of: laser ablation, drilling, milling, punching, chemical etching, and combinations thereof.
38. The insert according to item 37, wherein the openings are obtained by CO₂ laser ablation or UV laser ablation.
39. The insert according to any of items 37-38, wherein the first end wall is essentially planar at the edges of the openings.
40. A kit of parts comprising the insert according to any of items 1-39, and optionally a membrane precursor, and/or instructions to form a suspended membrane within the opening of the insert.
41. A method of producing the cell culture insert according to any of items 1-40 by a fabrication technique selected from the group consisting of: injection molding,
3D printing, casting, and thermoforming; and a cutting process selected from the group consisting of: laser ablation, drilling, milling, and punching, and optionally casting a suspended membrane within the at least one opening.
42. A method of producing a cell culture insert comprising the steps of:
providing a thermoplastic sheet (100), shaping the thermoplastic sheet into a chamber defined by side walls and a
first end wall by thermoforming into a mold, such as applying a vacuum to a mold (200),
cutting at least one opening in the first end wall,
optionally casting a suspended membrane within the at least one opening.
43. The method according to item 42 configured to produce the cell culture insert of any one of items 1-40.
44. The method according to any of items 42-43, wherein the sheet has a thickness of between 0.5-5 mm, such as 1.0, 1.5, or 2.0 mm, and preferably is above 1.0 mm.
45. The method according to any of items 42-44 wherein the thermoforming, such as the vacuum forming, is based on a female mold.
46. The method according to any of items 42-45, wherein the thermoforming, such as the vacuum forming, is carried out at a temperature above the glass transition of the thermoplastic sheet. 47. The method according to any of items 42-46, wherein the suspended membrane is casted based on a liquid membrane precursor, optionally a hydrogel precursor, such as a gelatine solution.
48. A method of forming a suspended membrane for supporting biomaterials, comprising the steps of:
providing a cell culture insert comprising a chamber defined by at least one side wall and a first end wall, wherein the first end wall has a predefined thickness equal to or below 500 μm and comprises at least one opening,
casting a suspended membrane within the at least one opening.
49. The method according to item 48, wherein the at least one opening is defined by an opening perimeter, and the casting is carried out within the opening perimeter, such that formed suspended membrane is supported at the opening perimeter by surface tension.
50. The method according to any of items 48-49, wherein the casting is based on a liquid membrane precursor, optionally a hydrogel precursor, such as a gelatine solution.
51. The method according to any of items 48-50, wherein the suspended membrane is formed by contacting the at least one opening with a liquid membrane precursor via dispensing the liquid, and/or by dipping into the liquid.
52. The method according to any of items 48-51, wherein the wherein the thickness of the suspended membrane is essentially similar to the predefined thickness of the first end wall, such as equal to or below 500 μm, more preferably equal to or below 200 μm, and most preferably equal to or below 100 μm, or such as above 100 μm and below 400 μm, more preferably between 110-350 μm or between 110-210 μm.
53. The method according to any of items 48-52, wherein the membrane is configured to support a culture of cells seeded onto the membrane, and/or supports culture of cells seeded into the membrane precursor.
54. The method according to item 53, where the cells are selected from the group consisting of: cancer cell lines, primary cell lines, stem cells, differentiated stem cells to a variety of cell types, tissue slices from biopsies, and combinations thereof.

1. A cell culture insert comprising a chamber defined by at least one side wall and a first end wall, wherein the first end wall has a thickness equal to or below 500 μm and comprises at least one opening, wherein the at least one opening is defined by an opening perimeter, the perimeter having a surface configured for supporting a suspended membrane at the edges of the membrane by surface tension.

2. The insert according to claim 1, wherein the perimeter surface does not comprise adhesives or glue.

3. The insert according to any of the preceding claims, wherein the first end wall is essentially planar with a predefined uniform thickness.

4. The insert according to any of the preceding claims, wherein the first end wall is essentially planar at the edges of the openings, such that the height of the perimeter surface is similar to the predefined uniform thickness of the first end wall.

5. The insert according to any of the preceding claims, wherein the first end wall comprises or consists of a hydrophilic material, such as polystyrene.

6. The insert according to any of the preceding claims, wherein the first end wall has a thickness of between 5-300 μm, more preferably between 10-250 μm, and most preferably between 15-200 μm, such as 20, 50, 70, 90, or 100 μm.

7. The insert according to any of the preceding claims, wherein the cross sectional area of the openings is between 0.1-100% of the area of the first end wall, more preferably between 1-95%, and most preferably between 10-90%.

8. The insert according to any of the preceding claims, wherein the shape of the openings in cross sectional view is selected from the group of: approximately circular, oval, ellipsoid, or polygonal such as squared or rectangular.

9. The insert according to claim 8, wherein the openings have a diameter of between 10-10.000 μm, more preferably between 50-5.000 μm, and most preferably between 100-1.500 μm, such as 250, 500, 750, or 1.000 μm.

10. The insert according to any of the preceding claims, wherein the thickness of the side walls is at least partially tapered

11. The insert according to any of the preceding claims, wherein at least a portion of the side walls form a cylinder or a hollow truncated cone.

12. The insert according to any of the preceding claims, dimensionally configured for insertion into a multi well plate.

13. The insert according to any of the preceding claims, wherein the opening comprises a suspended membrane within an opening cavity.

14. The insert according to claim 13, wherein the suspended membrane is supported at the edges of the membrane.

15. The insert according to any of claims 13-14, wherein the membrane comprises a hydrogel, optionally selected from the group of: gelatin, collagen, Matrigel®, alginate, agarose, fibrin gels, peptide gels, and any combinations thereof.

16. The insert according to any of claims 13-15, wherein the membrane has a porosity of above 40%, preferably between 50-99%, such as 70, 80, 85, or 95%.

17. The insert according to any of claims 13-16, wherein the thickness of the membrane is essentially similar to the thickness of the first end wall, such as equal to or below 500 μm, more preferably equal to or below 200 μm, and most preferably equal to or below 100 μm.

18. The insert according to any of claims 13-17, wherein the thickness of the membrane is essentially similar to the predefined thickness of the first end wall.

19. The insert according to any of claims 13-18 wherein the membrane supports culture of cells seeded onto the membrane, and/or supports culture of cells seeded into the membrane precursor.

20. A kit of parts comprising the insert according to any of claims 1-19, and optionally a membrane precursor, and/or instructions to form a suspended membrane within the opening of the insert.

21. A method of producing a cell culture insert comprising the steps of:
providing a thermoplastic sheet,
shaping the thermoplastic sheet into a chamber defined by side walls and a first end wall by thermoforming into a mold, such as applying a vacuum to a mold,
cutting at least one opening in the first end wall,
casting a suspended membrane within the at least one opening.

22. A method of forming a suspended membrane for supporting biomaterials, comprising the steps of:
providing a cell culture insert comprising a chamber defined by at least one side wall and a first end wall, wherein the first end wall has a predefined thickness equal to or below 500 μm and comprises at least one opening,
casting a suspended membrane within the at least one opening.

23. The method according to claim 22, wherein the at least one opening is defined by an opening perimeter, and the casting is carried out within the opening perimeter, such that formed suspended membrane is supported at the opening perimeter by surface tension.

24. The method according to any of claims 22-23, wherein the casting is based on a liquid membrane precursor, optionally a hydrogel precursor, such as a gelatine solution.

25. The method according to any of claims 22-24, wherein the suspended membrane is formed by contacting the at least one opening with a liquid membrane precursor via dispensing the liquid, and/or by dipping into the liquid.

26. The method according to any of claims 22-25, wherein the thickness of the suspended membrane is essentially similar to the predefined thickness of the first end wall.
